(19) United States
(12) Patent Application Publication
(10) Pub. No.: US 2024/0390249 A1
(43) Pub. Date: Nov. 28, 2024

(54) Materials and Methods for Inhibiting, Preventing, and Dispersing Biofilms

(71) Applicant: University of Wyoming, Laramie, WY (US)

(72) Inventors: Mark Gomelsky, Laramie, WY (US); Ahmed Elbakush, Laramie, WY (US); Alex M. Fulano, Nairobi (KE)

(73) Assignee: University of Wyoming, Laramie, WY (US)

(21) Appl. No.: 18/789,421

(22) Filed: Jul. 30, 2024

Related U.S. Application Data
(63) Continuation-in-part of application No. 17/878,517, filed on Aug. 1, 2022, now Pat. No. 12,048,309.
(60) Provisional application No. 63/227,732, filed on Jul. 30, 2021.
(60) Provisional application No. 63/533,292, filed on Aug. 17, 2023.
(60) Provisional application No. 63/638,144, filed on Apr. 24, 2024.

(51) Int. Cl. A61K 8/49; A61Q 11/00

(52) U.S. Cl. CPC A61K 8/4973; A61Q 11/00; A61K 2800/28; A61K 2800/59; A61K 2800/92

(57) ABSTRACT

Materials and methods are provided for preventing and dispersing biofilms. Methods and compositions are described for preventing and treating dental caries. Materials and methods are provided for producing antibiofilm agents from natural products, including maple (Acer sp.). Compositions are provided for inhibiting Sortase A activity in Bacillota.

### CROSS-REFERENCE TO RELATED APPLICATIONS

The present application is a continuation-in-part of U.S. application Ser. No. 17/878,517, filed Aug. 1, 2022, which claims the benefit of U.S. Application 63/227,732, filed on Jul. 30, 2021. The present application claims the benefit of U.S. Application 63/533,292, filed Aug. 17, 2023, and U.S. Application 63/638,144, filed Apr. 24, 2024. The entire contents of each of these is incorporated by reference herein, in its entirety, for all purposes.

### STATEMENT REGARDING FEDERALLY SPONSORED RESEARCH

This invention was made with government support under grant number 2020-67014-32496, (USDA-NIFA-AFRI-2020-67014-32496), awarded by the United States Department of Agriculture, National Institute of Food and Agriculture, and in part by the NIFA HATCH Program via the University of Wyoming Agriculture Experimental Station, grant WYO-00635-20. The government has certain rights in this invention.

### BACKGROUND

Bacteria can form aggregates rich in extracellular substances, or biofilms, that pose formidable challenges in medicine and agriculture because of their resistance to antibiotics, disinfectants, desiccation, and other treatments. Biofilms contribute to diseases, such as foodborne illness and dental carries. Biofilms increase tolerance of pathogenic bacteria to desiccation, disinfectants, and acids. Prevention and treatment options for these biofilms and associated pathogens can be limited by factors including: safety considerations, cost, consumer preference, and processing constraints.

As an example, fresh fruits and vegetables have been implicated in deadly outbreaks of listeriosis, caused by the bacterium Listeria monocytogenes. Avoiding fresh produce contamination altogether is difficult because L. monocytogenes is ubiquitously present in the environment, and some prevention measures, such as heat treatment and chemical disinfectants, are not suitable to treat fresh produce. Further, a recent metagenomic analysis of fecal matter revealed that significant percentages of farm animals and, surprisingly, humans carry L. monocytogenes asymptomatically. This represents an underappreciated source of contamination. While L. monocytogenes is omnipresent in the environment, most known fresh produce contamination incidents take place post-harvest, at the storage and processing facilities. The cleaning and disinfection protocols implemented at such facilities may be inadequate in removing L. monocytogenes, especially if it forms biofilms on the fresh produce or on the hard-to-access pieces of processing equipment. To eradicate L. monocytogenes biofilms, cleaning and disinfection is often insufficient, and application of specific antibiofilm agents may be advisable.

As another example, Streptococcus mutans, attaches to teeth surfaces where it forms biofilms or plaques. Acids generated by the bacteria decay enamel, resulting in the formation of cavities and can lead to serious infections. Some biofilm treatment measures, such as high heat or harsh disinfecting chemicals, cannot be used in treating an infected patient. Thus, there is a need for new and improved means for degrading biofilms, and for inhibiting or preventing biofilm formation.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like reference numerals designate corresponding parts throughout the views.

The patent or application file contains at least one drawing executed in color. Copies of this patent or patent application publication with color drawing(s) will be provided by the Office upon request and payment of the necessary fee.

FIG. 1 Effects of inhibitors on the Streptococcus mutans SrtA activity. The concentrations of inhibitors was 40 μM. NTG, nortrachelogenin-8′-O-beta-D-glucopyranoside; LR, lariciresinol; ECG, epicatechin gallate; EGCG, epigallocatechin gallate.

FIG. 2 Images of false teeth incubated for 48 h in liquid medium with two different strains of S. mutans (A, B), in the presence (+maple) or absence (−maple) of the 1:200 diluted maple syrup.

FIG. 3 Maple compounds with anti-SrtA activity inhibit EPS formation. Dispersion of exopolysaccharide (EPS)-biofilms (clumps) of the L. monocytogenes by the SrtA inhibitors: isoscopoletin (IS), abscisic acid (AA), epicatechin gallate (ECG). Absorbance (A₆₀₀) of bacterial culture is measured at the end of the 48-h incubation in the presence of the indicated compounds. Lower panels show representative images of the appearance of bacterial cultures after the incubation.

FIG. 4 L. monocytogenes sortase A is the target of maple compounds. SrtA inhibition by various maple compounds in vitro is shown. Concentrations (in μM) are shown in the legends.

FIG. 5 Formation of listerial EPS-biofilms on various fruit and vegetables is inhibited by the aqueous maple wood extract compared with control samples.

FIG. 6A-FIG. 6B The effect of maple compounds on L. monocytogenes biofilm inhibition at a range of maple syrup dilutions.

FIG. 7 Antibiofilm activity of nortrachelogenin-8′-O-beta-D-glucopyranoside (NTG) in L. monocytogenes.

FIG. 8 Antibiofilm activity of of lariciresinol in L. monocytogenes.

### DETAILED DESCRIPTION

Provided are biofilm control compositions and methods to reduce bacterial colonization, to inhibit biofilm formation, and to disperse biofilms. The methods and compositions include active agents that are nontoxic and can be derived from wood biomass. The methods and compositions include active agents that include sortase A inhibitors. The compositions can be formulated as cleaning compositions. The compositions can be formulated as a food additives suitable for ingestion. The compositions can be formulated as contact treatments and may be used to prevent biofilm formation on food articles, food processing equipment, food-contacting surfaces, medical instruments, and personal care items. The compositions can be formulated as personal care products for topical use. The compositions can be formulated as oral care products to treat, mitigate, or prevent oral caries, and other diseases associated with biofilm-forming bacteria.

Materials and methods to inhibit or treat disease caused by pathogens including biofilm-forming bacteria are described. Antibacterial and antibiofilm agents are provided. These agents can include agents that disperse bacterial biofilms. These agents can include agents that inhibit bacterial biofilm formation, bacterial attachment, bacterial biofilm persistence, bacterial persistence, and/or bacterial infection. Selected antibiofilm agents can inhibit sortase A (SrtA), an enzyme involved in anchoring surface receptors to the bacterial cell wall and in facilitating attachment of the bacterial cells to biotic or abiotic surfaces.

A composition for inhibiting or dispersing biofilms can include an antibiofilm-effective amount of an active compound. The active compound can include one or more of: nortrachelogenin 8′-O-β-D-glucopyranoside (NTG), lariciresinol, scopoletin, isoscopoletin, epicatechin gallate, abscisic acid, or mixtures thereof. The composition can include a carrier or vehicle.

An oral care composition can include an antibiofilm-effective amount of an active compound. The oral care composition can include an orally-acceptable vehicle and a sweetener. The active compound can include one or more of: NTG, lariciresinol, scopoletin, isoscopoletin, epicatechin gallate, or abscisic acid.

A composition can comprise an extract obtained from biomass of a member of the genus Acer. The extract can comprise an active agent having antibiofilm properties, which can inhibit biofilm formation, disperse biofilms, and prevent attachment to biotic and abiotic surfaces by pathogenic bacteria.

In an example, a composition for preventing or dispersing a biofilm is provided. In an example, the biofilm is produced by at least one bacterial strain belonging to the branch Bacillota and containing a SrtA enzyme, wherein the bacterial strain is in a genus including at least one of: Streptococcus, Staphylococcus, Enterococcus, Clostridia, Lactobacillus or Listeria.

The active compound can be in, or produced from, an extract of maple, pecan or hickory. The compositions may include an aqueous extract from wood biomass. The compositions may include an extract from wood biomass of a maple tree (Acer sp.). The compositions may include an extract from wood biomass of a hickory or pecan tree (Carya sp.).

In an example, the wood biomass includes at least one of: heartwood, sapwood, pith, twigs, leaves, stems, branches, nuts, nut shells, sap, roots, or bark. In an example, the wood biomass comprises wood chips that include 30% to 80% heartwood and 20% to 70% sapwood. In an example, the wood chips include tree wood derived from a portion of a tree having a diameter greater than 5 cm. In an example, the wood biomass comprises sap.

In an example, an extract of wood biomass is derived from maple wood. In an example, the maple is an Acer tree selected from: Acer nigrum, Acer lanurn, Acer acuminatum, Acer albopurpurascens, Acer argutum, Acer barbinerve, Acer buergerianum, Acer caesium, Acer campbeffii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer x peronai, Acer pictum, Acer pilosum, Acer platanoides, Acer poliophyllum, Acer x pseudoheldreichii, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, or hybrids thereof.

In an example, the maple is a hard maple including at least one of: sugar maple, Acer saccharum, or black maple, Acer nigrum. In an example, the maple is a soft maple including at least one of: bigleaf maple (Acer macrophyllum), red maple (Acer rubrum), silver maple (Acer saccharinum), or boxelder (Acer negundo).

In an example, the wood biomass is derived from hickory. In an example, the hickory includes at least one of: Shagbark Hickory (Carya ovata), Bitternut Hickory (Carya cordiformis), Mockernut Hickory (Carya tomentosa), Nutmeg Hickory (Carya myristiciformis), Pecan (Carya illinoinensis), Pignut Hickory (Carya glabra), Shellbark Hickory (Carya laciniosa), or Water Hickory (Carya aquatica).

In some embodiments, the anti-biofilm composition comprises at least one phenolic or polyphenolic compound with antibiofilm activity. In some embodiments, at least one phenolic compound is derived or extracted from wood biomass. In some embodiments, at least one phenolic compound is a purified composition consisting essentially of at least one phenolic compound. In some embodiments, the purified composition includes a solvent or excipient. In some embodiments, the anti-biofilm composition comprises at least two phenolic compounds with antibiofilm activity. In some embodiments, at least one phenolic compound is lariciresinol, scopoletin, isoscopoletin, epicatechin gallate, or abscisic acid. In some embodiments, the anti-biofilm composition comprises nortrachelogenin 8′-O-beta-D-glucopyranoside and at least one of: lariciresinol, scopoletin, or abscisic acid. In some embodiments the anti-biofilm composition is characterized by an effective amount of at least two phenolic compounds having anti-biofilm activity. In some embodiments the anti-biofilm composition is prepared by combining and mixing a first solution and a second solution, wherein the first solution comprises at least one phenolic compound produced by extraction, and the second solution comprises at least one second phenolic compound, different from the first phenolic compound. In some embodiments, the at least one second phenolic compound is produced by extraction or by chemical synthesis. In some embodiments, the proportion or ratio of the active agents in the prepared composition differs from a proportion or ratio of the active agents or phenolic compounds in the wood biomass. In some embodiments, the anti-biofilm composition comprises at least three phenolic compounds with antibiofilm activity. In some embodiments, the anti-biofilm composition comprises a plurality of phenolic compounds with antibiofilm activity, wherein the combination is synergistic, whereby the combination is has greater anti-biofilm efficacy than a similar concentration of any one of the phenolic compounds.

Compositions can include one or more of: a surfactant, a buffer, a sweetener, a flavoring, a fragrance, a solvent, an enzyme, a stabilizer, an emulsifier, a humectant, an abrasive, a mineral source, a preservative, an antimicrobial, an anti-inflammatory, an analgesic, a coating enhancer, or a botanical constituent. The composition can exclusively consist of food-safe or GRAS ingredients.

In an example, a buffer or pH adjuster can include one or more of: ammonium bicarbonate, ammonium carbonate, ammonium citrate, ammonium hydroxide, ammonium phosphate, calcium carbonate, calcium chloride, calcium citrate, calcium fumarate, calcium hydroxide; calcium phosphate, magnesium carbonate, magnesium citrate, magnesium hydroxide, magnesium phosphate, magnesium sulfate, potassium bicarbonate, potassium carbonate, potassium citrate, potassium fumarate, potassium hydroxide, potassium sulfate, sodium bicarbonate, sodium carbonate, sodium citrate, sodium hydroxide, and sodium phosphate.

In an example, a sweetener can include one or more of: dextrose, polydextrose, sucrose, maltose, dextrin, dried invert sugar, mannose, xylose, ribose, fructose, levulose galactose, corn syrup, stevia, partially hydrolyzed starch, hydrogenated starch hydrolysate, sorbitol, mannitol, xylitol, maltitol, isomalt, cyclamates, dihydrochalcones, aspartame, neotame, sucralose, saccharin and salts thereof. In an example, a sweetener can include naturally-occurring sweetener, including for example: stevia, glycyrrhizin, or a sugar alcohol. Suitable sugar alcohols can include: xylitol, sorbitol, mannitol, maltitol, glycerol, lactitol, and erythritol.

In a preferred example, a sweetener for an oral care product may include xylitol, which has been found to contribute to tooth remineralization and suppress growth of some pathogenic bacteria, such as S. mutans. The combination of a sortase A inhibitor with xylitol may produce a synergistic benefit to oral heath.

A flavorant can include an orally-acceptable natural or synthetic flavoring, such as essential oils, flavoring aldehydes, flavoring oils, esters, and alcohols. In an example, a flavorant can include extracts or essences of one or more of: spearmint, peppermint, cinnamon, wintergreen, sassafras, clove, vanilla, caramel, honey, coffee, chocolate, cocoa, cola, tea, almond, ginger, nutmeg, eucalyptus, pine, cinnamon, fennel, bergamot, hyssop, rose, lavender, sage, marjoram, parsley, rosemary, licorice, lemon, lime, grapefruit, orange, apricot, plum, banana, grape, apple, strawberry, cherry, pineapple, and pear. Also useful are such chemicals as menthol, vanillin, thymol, limonene, citral, and anethole. Also encompassed within flavorants herein are ingredients that provide sensory effect in the mouth, including cooling or warming effects. Such ingredients can include one or more of: menthol, menthyl acetate, menthyl lactate, camphor, eucalyptus oil, cucalyptol, anethole, eugenol, cassia, oxanone, irisone, thymol, linalool, benzaldehyde, cinnamaldehyde. In some examples, the composition includes a flavorant including one or more of: menthol, vanillin, maple furanone, furancol, cinnamaldehyde, methyl cinnamate, or cinnamic acid.

In an example, an enzyme can include one or more of: lactoperoxidase, lysozyme, glucose oxidase, galactosidase, amylase, lactase, lipase, or PssZ enzyme. In an example, the composition comprises PssZ enzyme. U.S. Pat. No. 10,383,338 describes methods for making and using PssZ enzymes and is incorporated by reference herein. In some examples an enzyme may be stabilized by adsorption, covalent binding, crosslinking, entrapment, reverse micelleation, chemical modification, lyophilization, protein engineering, ionic liquid coating, or stabilizing additives.

In an example, a mineral or ion source can include one or more of: calcium lactate, sodium phosphate, potassium phosphate, dicalcium phosphate, calcium carbonate, stannous fluoride, sodium fluoride, or sodium monofluorophosphate.

In an example, the composition can include one or more antimicrobials and/or preservatives; these may include one or more of: citric acid, zinc citrate, zinc oxide, tocopherols, tocotrienols stannum salts, triclosan, sodium benzoate, methyl paraben, or ethyl paraben. In an example, the composition comprises an antifungal, such as natamycin.

In an example, the composition can include an anti-inflammatory. In an example, the composition can include an analgesic.

In an example, the composition can include a botanical constituent, which can include an extract, juice, decoction, or other product from one or more plants. In an example, the botanical constituent includes star jasmine, Trachelospermum jasminoides.

In an example, the composition further comprises a coating enhancer to modify the rheometric properties and viscosity of the composition. The coating enhancer may include starch, amylose, amylopectin, dextrin, maltodextrin, polydextrose, syrup, cellulose, gum Arabic, gum tragacanth, gum karaya, mesquite gum, galactomannan, pectin, carrageenan, alginate, dextran, xanthan, gellan, whey protein, silk protein, casein, gelatin, gluten, fatty acids, fatty alcohols, wax, beeswax, carnauba wax, candellia wax, glyceride, and phospholipid, PVP, paraffin, shellac, or a solgel. In an example method, the composition substantially coats a surface and forms a dried residue on the surface. In an example method, the composition may be applied to a natural or porous surface with a coating enhancer to form a biofilm-inhibiting coating, film, or residue on the surface.

In some examples, the composition includes one or more of: sorbitol, xylitol, stevia, quebecol, inulin, saccharin, aspartame, sucralose, glycerol, glycerin, silica, disodium phosphate, calcium phosphate, calcium glycerophosphate, titanium dioxide, xanthan gum, polyethylene glycol, sodium benzoate, benzoic acid, sodium fluoride, stannous fluoride, sodium monofluorophosphate, sodium tripolyphosphate, zinc chloride, potassium nitrate, citric acid, calcium carbonate, magnesium carbonate, sodium bicarbonate, hydroxyapatite, zeolite, mica, coconut oil, sodium lauryl sulfate, or carrageenan. In some examples the composition includes glycerin, also known as glycerol, which can function as a vehicle, sweetener, humectant, solvent, stabilizer, and viscosity modifier.

In an example, the anti-biofilm agent is provided in a treatment composition, and the composition comprises a liquid, an aqueous solution, an emulsion, a foam, a suspension, aerosolized droplets, or a spray.

A composition can comprise an extract obtained from a member of the genus Acer. The extract can comprise an agent having antibiofilm properties, which can inhibit biofilm formation, disperse biofilms, and prevent attachment to biotic and abiotic surfaces by pathogenic bacteria. The agent can comprise at least one sortase A (SrtA) inhibitor.

A method for dispersing a biofilm on a surface can comprising contacting the surface with a composition comprising at least one SrtA inhibitor. In an example, a method for dispersing a biofilm on a surface comprising contacting the surface with a composition comprising at least two agents having antibiofilm properties, wherein the agents are selected from the group consisting of: NTG, lariciresinol, scopoletin and isoscopoletin, epicatechin gallate, epigallocatechin gallate, or abscisic acid.

In some embodiments, the composition comprises a concentration of NTG at a level in a range from 0.5 mg/mL to 5 mg/mL. In some embodiments the concentration of NTG in the composition is greater than 0.3 g/L, greater than 0.4 g/L, greater than or equal to 0.5 g/L, greater than or equal to 0.7 g/L, greater than or equal to 0.9 g/L, greater than or equal to 1.0 g/L, greater than or equal to 1.5 g/L, greater than or equal to 2.0 g/L, greater than or equal to 2.5 g/L, greater than or equal to 3.0 g/L, greater than or equal to 3.5 g/L, greater than or equal to 4.0 g/L, or greater than or equal to 4.5 g/L. In some embodiments the concentration of NTG in the composition is less than 7.0 g/L, less than 6.5 g/L, less than to 6.0 g/L, less than or equal to 5.5 g/L, less than or equal to 5.0 g/L, less than or equal to 4.5 g/L, less than or equal to 4.0 g/L, less than or equal to 3.5 g/L, less than or equal to 3.0 g/L, or less than or equal to 2.5 g/L.

The structure of nortrachelogenin 8′-O-β-D-glucopyranoside, also known as nortrachelogenin-8′-O-beta-glucoside, and also referred to as NTG, may be illustrated as:

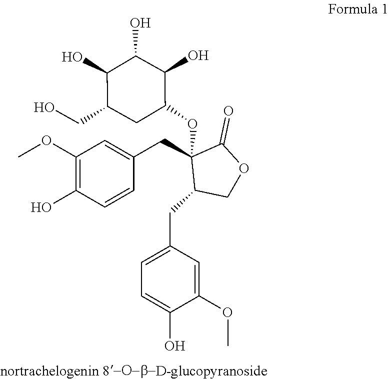

A composition can comprise an active agent having antibiofilm properties. The active agent can comprise at least one SrtA inhibitor. In an example, a method of treating a S. mutans infection in a patient comprises administering to the patient an effective amount of a maple compound capable of inhibiting SrtA.

Prevention of biofilm formation and treatment of diseases associated with bacterial biofilms is relevant to many areas of healthcare, hygiene, cosmetics, and animal care.

Provided are methods for dispersing a biofilm on a surface, preventing biofilm formation on a surface, or treating a disease associated with a bacterial biofilm, the method comprising contacting a composition comprising an agent to the surface. In some examples, the surface includes a dermal surface. In some examples, the surface includes a mucous membrane. In some examples, the surface includes at least a portion of a mouth or oral surface including at least a portion of: a buccal surface, a gingival surface, a dental surface, a lingual surface, or a palate surface. In some examples, the agent comprises at least one SrtA inhibitor. In some examples, the agent comprises at least two SrtA inhibitors. In some examples, the agent comprises at least three SrtA inhibitors. In an example, a SrtA inhibitor is selected from: nortrachelogenin-8′-O-β-D-glucopyranoside, lariciresinol, scopoletin, isoscopoletin, epicatechin gallate, epigallocatechin gallate, or abscisic acid. In an example, the agent is derived from maple biomass and comprises at least one of: nortrachelogenin-8′-O-β-D-glucopyranoside, lariciresinol, scopoletin, isoscopoletin, epicatechin gallate, or abscisic acid. In an example, the agent is derived from a maple sap, aqueous extract of maple wood, or maple syrup.

In some examples, the composition is prepared from wood biomass, wherein the wood is maple (Acer sp.). In some examples, the composition is prepared from an aqueous wood extract. In some examples, the composition is prepared from sap or syrup. In some examples, the composition is prepared from sap or syrup and the preparation process includes removing at least a portion of sucrose. In some examples, the composition comprises a sugar-depleted maple syrup. In some examples, the composition comprises an aqueous solution having between 0.0001% to 15.0% sucrose by weight. In some examples, the composition comprises a sugar-depleted aqueous solution having less than 10% sucrose by weight. In some examples, the composition comprises a sugar-depleted extract having sucrose in a range from 0.001% to 15.0% by weight. In some examples, the composition comprises a sugar-depleted aqueous solution having less than 10.0%, less than 5.0%, less than 2.0%, less than 1.0%, less than 0.5%, less than 0.1%, or less than 0.01%, w/w sucrose.

In some examples, the agent is provided in the composition at a concentration equal to or greater than 5.0 microMolar (μM), greater than 10 μM, greater than 20 μM, greater than 50 μM, greater than 60 μM, greater than 100 μM, greater than 120 μM, greater than 150 μM, greater than 200 μM, in a range up to 250 μM, in a range up to 500 μM, in a range up to 750 μM, in a range of 5 μM to 1000 μM, or in a range of 5 μM to 2500 μM. In some examples, the agent is at least one of: NTG, LR, S, IS, LR, or ECG, and is provided in the composition at a concentration in a range of 5 μM to 200 μM, in a range of 10 μM to 150 μM, or in a range of 20 μM to 80 μM. In some examples, the agent is AA, and is provided in the composition at a concentration in a range of 50 μM to 500 μM.

In some examples, methods include contacting the composition to the surface for a contact time greater than 1 minute. In some examples, methods include contacting the composition to the surface for a contact time in a range of 0.5 minute to 60 minutes. In some examples, methods include contacting the composition to the surface for a contact time in a range of 0.5 minute to 60 minutes, and repeating the contacting at least once per day for 3-6 weeks. In some examples, methods include contacting the composition to the surface for a contact time and repeating the contacting 1 to 14 times per week for 2-8 weeks, or longer.

Methods are provided for prevention of biofilm formation and treatment of diseases associated with bacterial biofilms. In some examples, the methods are directed to a pathogen comprising at least one bacterial strain in a genus including at least one of: Streptococcus, Staphylococcus, Enterococcus, Clostridia, Lactobacillus, or Listeria. In an example, the pathogen is Streptococcus mutans. In another example, the pathogen is Staphylococcus aureus.

In an example, a method of treating an infection in a patient by a sortase-A-producing organism in the Bacillota phylum comprises: administering to the patient an effective amount of an agent capable of inhibiting sortase A. In some examples, a method of treating a S. mutans infection in a patient comprises administering to the patient an effective amount of an agent capable of inhibiting sortase A, at a dose sufficient to inhibit bacterial cell aggregation.

### Compositions for Oral Care are Provided

An oral care composition can comprise an aqueous solution including a plurality of SrtA inhibiting agents. In an example, a composition for oral care can be provided in the form of a mouthwash. The mouthwash can include an aqueous carrier; a sweetener, such as xylitol; and one of more SrtA inhibiting agents derived from maple. A first SrtA inhibiting agent can include NTG provided in the composition at a concentration in a range of 20 μM to 200 μM. A second SrtA inhibiting agent can include lariciresinol provided in the composition at a concentration in a range of 20 μM to 200 μM. A third SrtA inhibiting agent can include scopoletin provided in the composition at a concentration in a range of 20 μM to 200 μM. A fourth SrtA inhibiting agent can include isoscopoletin provided in the composition at a concentration in a range of 20 μM to 200 μM. A fifth SrtA inhibiting agent can include epicatechin gallate provided in the composition at a concentration in a range of 20 μM to 200 μM. A sixth SrtA inhibiting agent can include epigallocatechin gallate provided in the composition at a concentration in a range of 20 μM to 200 μM. A seventh SrtA inhibiting agent can include abscisic acid, including derivatives thereof, provided in the composition at a concentration in a range of 20 μM to 200 μM. In some examples the abscisic acid, including derivatives thereof, includes one or more of: (+) abscisic acid, (−) abscisic acid, 7-hydroxyabscisic acid, trans-abscisic acid, phaseic acid, neo-phaseic acid, or dihydrophaseic acid.

An oral care composition can comprise an extract obtained from a member of the genus Acer. The extract can comprise an agent having antibiofilm properties, which can inhibit biofilm formation, disperse biofilms, and prevent attachment to oral surfaces by pathogenic bacteria, such as Streptococcus mutans, when introduced to the oral cavity. The agent can comprise at least one sortase A (SrtA) inhibitor.

The composition may take a form useful for oral administration. Illustrative examples include: a mouthwash, mouth rinse, or mouth spray; a dentifrice, such as, a toothpaste, dental gel, dental cream, dental varnish, or tooth powder; a gum or confectionary; a lozenge; dental floss or dental tape; a prophylaxis paste or powder; an oral film or gel strip, such as, tooth strips or breath strips, preferably using a biodegradable or orally-consumable film or gel; denture adhesives; or a coating on an oral appliance or orthodontic implant.

Provided are antiplaque oral compositions. Methods are provided for inhibiting dental plaque and promoting caries reduction in oral hygiene. In some examples, the agent is provided in a dental care product. In some examples, the agent is provided in a composition for administering as a mouthwash (an oral rinse). In an example treatment method, a composition including the anti-biofilm agent is contacted to an oral surface by spraying, swabbing, coating, soaking, or rinsing. In some examples the antibiofilm agent is ingested after contact with an oral surface.

In an example, the oral care composition comprises a maple extract and the maple extract comprises an antibiofilm agent including one or more of: nortrachelogenin-8′-O-beta-D-glucopyranoside (NTG), lariciresinol (LR), scopoletin, isoscopoletin (IS), epicatechin gallate (ECG), or abscisic acid (AA). In some examples, the extract includes at least two of the antibiofilm agents in an aqueous solution at a concentration in a range of 20.0 μM to 200 μM.

In an example, the oral care composition formulation comprises: 0.5 to 15 wt. % surfactant, 10 to 98 wt. % aqueous carrier, 0.1 to 10.0 wt. % sweetener, 0.1 to 10.0 wt. % humectant, and 0.01 to 2.00 wt. % of a maple-derived extract or blend comprising active agents.

In some examples, the agent is provided in a dental care product selected from the group comprising: candy, lozenge, chewing-gum, lollipop, chewable tablet, gelatin-gum, chew toy, wafer, capsule, toothpaste, tooth gel, prophylactic paste, toothpowder, mouthwash, mouth-spray, solution, coated dental floss, coated interdental brush, coated toothbrush, retainer cleaning tablet, denture cleaner, or oral swab. In an example, the composition exclusively consists of food-safe ingredients.

In some embodiments, the dental care product includes one or more of: a sweetener, a dispersing agent, a surfactant, an antimicrobial agent, a flavoring agent, or a fluoride source. In some embodiments the dental care product includes a remineralization promoter, such as fluoride, a calcium ion source such as calcium lactate, hydroxyapatite, or a phosphate ion source such as monobasic sodium phosphate.

Examples include a pharmaceutical composition in the form of a toothpaste or mouthwash for the treatment of a S. mutans infection which comprises a pharmaceutically acceptable carrier and an effective amount of an antibiofilm substance comprising at least one SrtA inhibitor agent. In some examples, the composition comprises a plurality of SrtA inhibitor agents. In some examples, the composition comprises a plurality of SrtA inhibitor agents, including NTG, LR, IS, and ECG.

In some examples, a dental care product is provided for pets or other domestic animals. In some examples, a dental care product includes the agent in a pet chew treat, food, toy, or drinking additive.

### Compositions for Personal Care and Medical Use are Provided

In some examples, maple-derived compounds are used in the treatment of nasal cavities to inhibit accumulation of Staphylococcus aureus and other pathogenic staphylococci. In some examples, an agent capable of inhibiting a sortase A (SrtA) enzyme is formulated for administration by nasal spray, nasal rinse, neti pot, nebulizer, respirator, aerosol, or inhaler. In an example, the agent is provided as an aqueous solution in a humidifier of a positive airway pressure device.

In some examples, a method of treating a skin disorder in a patient comprises administering to the patient an effective amount of an agent capable of inhibiting sortase A (SrtA) enzyme. In some examples the agent is applied topically by contacting a composition comprising the agent to the skin. In some examples, the skin disorder is acne. In some examples, the skin disorder is S. aureus-induced pimples. In some examples the composition is formulated as a cream, ointment, gel, mask, balm, scrub, wipe, spray, lotion, or foam.

In some examples, maple-derived compounds are used in wound treatment to prevent Streptococcus and/or Staphylococcus from attaching to wound surfaces and prevent forming biofilms.

Bacterial endocarditis infections can be associated with Enterococcus sp., Streptococcus Sp., Listeria sp., or Staphylococcus sp. In some examples, a method of treating a bacterial endocarditis infection in a patient comprises administering to the patient an effective amount of an agent derived from maple biomass capable of inhibiting sortase A (SrtA) enzyme.

Dental caries is associated with elevated risk of some systematic diseases and conditions, including heart disease. Bacteria present in the oral cavity, may gain access to distant sites in the body through the circulatory and respiratory system, with the potential to cause adverse health conditions. In some examples, a prophylactic method for preventing or treating a bacterial infection in a patient comprises administering to the patient an effective amount of an agent capable of inhibiting sortase A (SrtA) enzyme. In some examples, the treatment is administered topically to an oral surface. In some examples, the treatment is administered within a 24 hour period preceding oral surgery. In some examples, the treatment is administered to a patient presenting at least one of: pericoronitis, periodontitis, gingivitis, stomatitis, sinusitis, sepsis, oral abscess, pulp necrosis, orofacial granulomatosis, oral Crohn's disease, Behçet's disease, aphthous stomatitis, odontogenic infection, or periodontic-endodontic lesions.

In some examples, a method of treating infection in a patient comprises administering to the patient an effective amount of an agent capable of inhibiting sortase A enzyme. In some examples, the infection is caused by at least one strain of SrtA-containing bacteria, including, for example, one or more of Streptococcus, Staphylococcus, or Enterococcus.

### Compositions for Cleaning and Food Treatment are Provided

In an example treatment method, the composition is applied to a food product by spraying, soaking, or rinsing. In an example, produce is soaked in an aqueous solution of the composition for a period in a range of 1 to 60 minutes. In an example, leafy greens are sprayed with the composition prior to packaging into a packaged salad product.

In an example treatment method, the composition is applied to a porous surface and the porous surface is part of a food container, a food-contacting surface, or a surface in a food production facility. In embodiments, the food contacting surface comprises a porous material such as wood, wicker, rope, paper, cardboard, bamboo, sponge, or cloth. In embodiments, the porous material is a plant-based natural material. In an example, the food contacting surface includes at least one of: a bin, a crate, a box, a tray, a mat, a spout, a bucket, a cloth, a cheesecloth, a cutting board, a net, a bag, a sponge, a basket, a carton, a countertop, or packing material.

Prevention of pathogenic biofilm formation, particularly by L. monocytogenes, is relevant to the dairy industry and to packaged food producers. In an example treatment method, a solution comprising the active agent is applied to surfaces in a food processing facility. In an example treatment method, a solution comprising the anti-biofilm agent is provided in a boot wash tray for use by workers in a food processing facility. In an example, the food processing facility is a dairy processing facility or a produce packaging facility. In an example treatment method, a solution comprising the anti-biofilm agent is applied to hay, straw, silage, animal fodder, animal bedding, or an animal stall. In another example treatment method, a composition is applied to skin, for example, to the hands of an animal handler, to the hide of an animal, or to the udders of an animal. In an example, the anti-biofilm agent is incorporated into an emulsion, a suspension, a foam, or a cream.

Methods of using and producing the antibiofilm agents are provided. Formulations and methods are provided for preventing, inhibiting, controlling, and/or treating bacterial disease. Methods for modulating biofilm dispersal in oral pathogens, can include contacting the pathogen with a composition comprising the agent.

### Experimental Studies, Results, and Discussion

Experimental studies were performed, testing the effect of maple-derived compositions on selected bacterial strains and bacterial biofilms. Experimental data indicate that the maple-derived compositions can be used to inhibit, prevent, and disperse biofilms. Effects of the maple compositions were demonstrated for multiple bacterial species, including organisms from genera: Listeria and Streptococcus. Representative experimental results are summarized and discussed below.

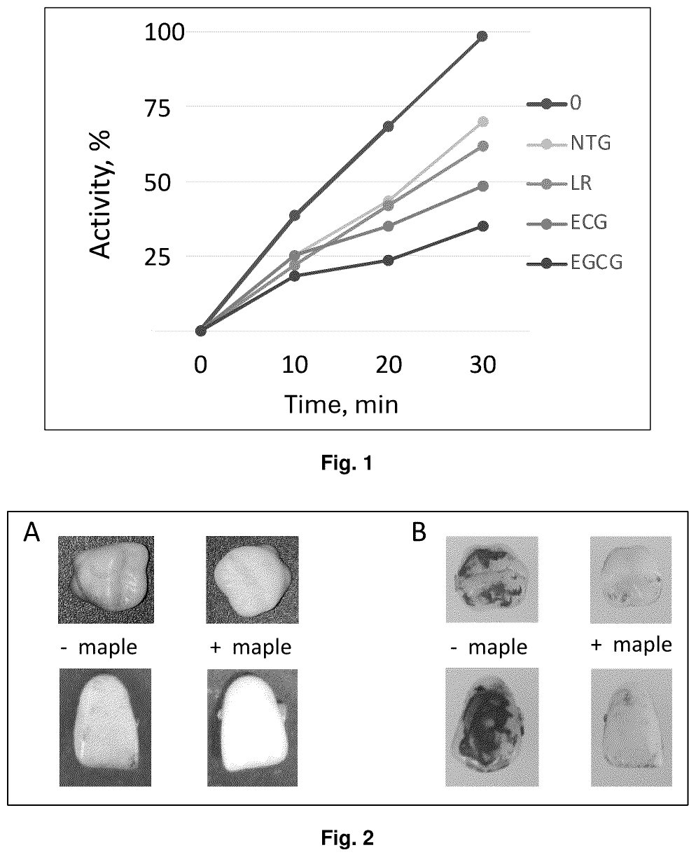

FIG. 1 shows effects of inhibitors on the S. mutans SrtA activity. The inhibitors included nortrachelogenin-8′-O-β-D-glucopyranoside (NTG), lariciresinol (LR), epicatechin gallate (ECG), Epigallocatechin gallate (EGCG); the concentrations of inhibitors was 40 μM.

FIG. 2 shows images of false teeth incubated for 48 h in liquid medium with two different strains of S. mutans (A, B), in the presence (+maple) or absence (−maple) of the 1:200 diluted maple syrup. Teeth were removed, rinsed in water and stained in Crystal violet to reveal attached biofilms.

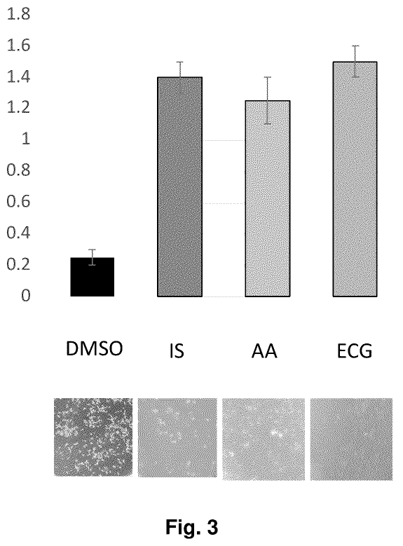

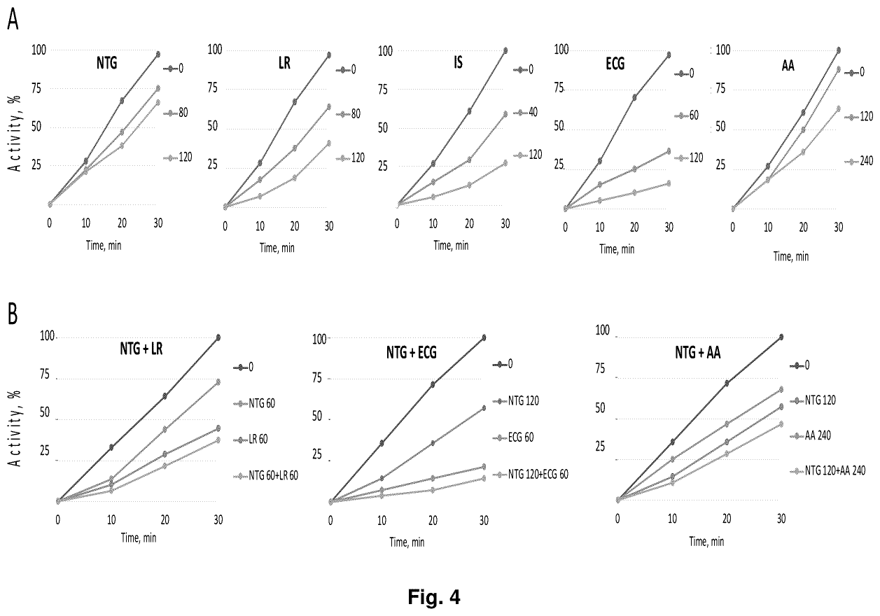

FIG. 3 and FIG. 4 show inhibition of Sortase A promotes dispersion of biofilms. Isolated compounds of maple products cach individually inhibit biofilm formation and disperse biofilms. FIG. 3 shows that maple compounds with anti-SrtA activity inhibit formation of the EPS-biofilms in L. monocytogenes. The L. monocytogenes SrtA inhibitors tested include: isoscopoletin (IS), abscisic acid (AA), epicatechin gallate (ECG). Absorbance (A₆₀₀) of bacterial culture is measured at the end of the 48-h incubation in the presence of the indicated compounds. Lower panels show representative images of the appearance of bacterial cultures after 48-h incubation in the presence of the respective compounds. FIG. 4, shows activity of the purified membrane-less SrtA::His₆, which was tested using the fluorescently labelled LPxTG-containing peptide substrates. Concentrations (in μM) are shown in the legends. Combinations of maple compounds increased Sortase A inhibition. Tested compounds included: nortrachelogenin-8′-O-β-D-glucopyranoside (NTG); lariciresinol (LR); isoscopoletin (IS); epicatechin gallate (ECG); abscisic acid (AA); NTG plus LR; NTG plus ECG; NTG plus AA.

Additional compounds were tested for activity against EPS biofilms formed by L. monocytogenes in various other assays. Some of these compositions included: catechin, epicatechin, procyanidin A2, epicatechin gallate, nortrachelogenin (also known as pinopalustrin), and NTG.

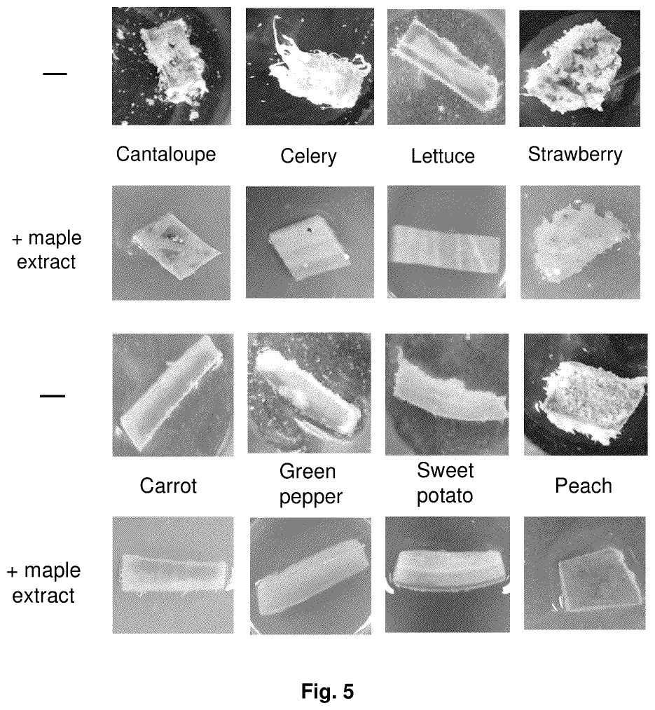

FIG. 5 shows maple-derived compounds prevented biofilm formation by L. monocytogenes on various pieces of fruit and vegetables, and induced dispersion of existing biofilms.

Inhibitory effects were shown for maple products. Inhibition of cell aggregation was demonstrated for maple compositions sourced from wood biomass, sap, and syrup. Maple products inhibiting cell aggregation in L. monocytogenes possess the anti-SrtA activity in vitro.

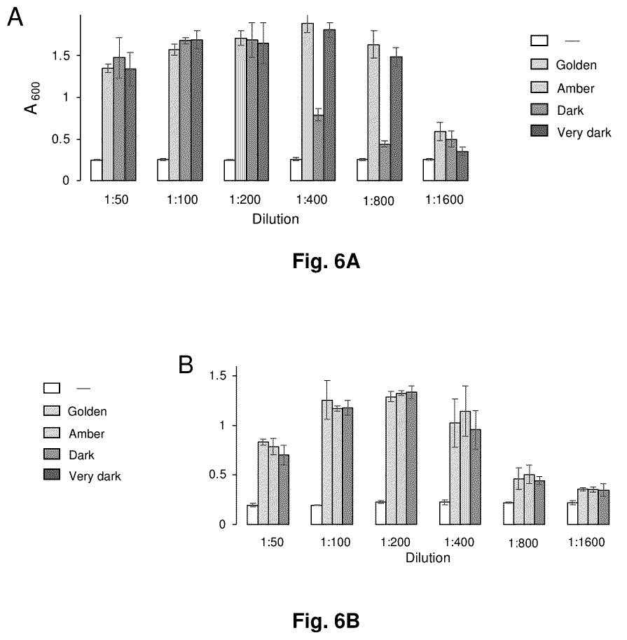

FIGS. 6A-6B show inhibition of cell aggregation was demonstrated for maple compositions at a range of dilutions (1:50 to 1:1600). The effect of maple syrup compounds on L. monocytogenes biofilm inhibition at a range of dilutions is shown.

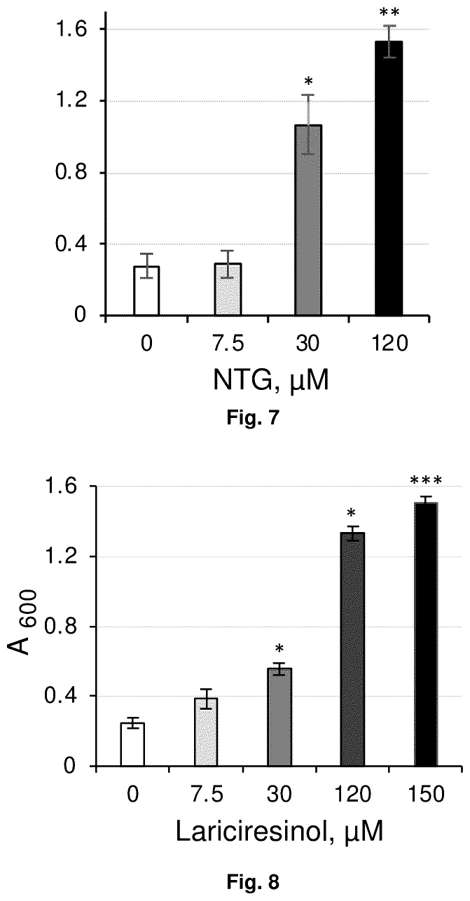

FIG. 7 illustrates L. monocytogenes antibiofilm activity of NTG.

FIG. 8 illustrates L. monocytogenes antibiofilm activity of lariciresinol.

Molecular modeling indicated mechanisms of action relating to binding modes of selected maple compounds with amino acids of the Sortase A enzyme, including both hydrophilic (H-bonds) and hydrophobic binding sites. The molecular models showed that the tested maple compounds possessing anit-SrtA activity in vitro bind in the catalytic site of SrtA with negative Gibbs free energy (ΔG) values in the range of 5.0-7.4 kcal/mol (Table 1). The most potent SrtA inhibitor from maple in vitro, ECG, has the highest absolute ΔG value. Some SrtA inhibitors may have second binding sites with the negative ΔG values outside of the catalytic site of SrtA. These additional interactions may stifle the conformational flexibility of SrtA and enhance inhibition. Binding at multiple sites may help “lock” SrtA in the inactive conformation more efficiently. Synergistic effects may exist with other combinations of inhibitors or with combinations of SrtA inhibitors and non-inhibitory maple compounds

TABLE 1
Predicted ΔG of binding of SrtA inhibitors.

| Compound | ΔG, kcal/mol |
|---|---|
| Nortrachelogenin-8′-O-β-D-glucoside (NTG) | −7.2 |
| Lariciresinol (LR) | −6.2 |
| Isoscopoletin (IS) | −5.0 |
| (−)Epicatechin gallate (ECG) | −7.4 |
| Abscisic acid (AA) | −5.9 |

Certain embodiments of the devices, apparatuses, and methods disclosed herein are defined in the above examples. It should be understood that these examples, while indicating particular embodiments, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure, and to adapt a particular situation or material to the teachings of the disclosure without departing from the essential scope thereof.

What is claimed is:

1. An oral care composition comprising:
a carrier consisting essentially of an aqueous liquid;
a first SrtA inhibiting agent provided in the aqueous liquid at a concentration in a range of 20 μM to 1000 μM, wherein the first SrtA inhibiting agent is selected from the group consisting of: nortrachelogenin-8′-O-β-D-glucopyranoside, lariciresinol, scopoletin, isoscopoletin, epicatechin gallate, epigallocatechin gallate, or abscisic acid;
a second SrtA inhibiting agent provided in the aqueous liquid at a concentration in a range of 20 μM to 1000 μM, wherein the second SrtA inhibiting agent is selected from the group consisting of: nortrachelogenin-8′-O-β-D-glucopyranoside, lariciresinol, scopoletin, isoscopoletin, epicatechin gallate, epigallocatechin gallate, or abscisic acid; and wherein the second SrtA inhibiting agent is different from the first SrtA inhibiting agent; and
a sweetener comprising at least one of xylitol, sorbitol, mannitol, or erythritol;
wherein the composition contains less than 1.0 g/L sucrose; and
wherein the composition is formulated as a liquid mouthwash, oral spray, or oral rinse.

2. The oral care composition of claim 1, further comprising:
a third SrtA inhibiting agent provided in the aqueous liquid at a concentration in a range of 20 μM to 1000 μM, wherein the third SrtA inhibiting agent is selected from the group consisting of: nortrachelogenin-8′-O-β-D-glucopyranoside, lariciresinol, scopoletin, isoscopoletin, epicatechin gallate, epigallocatechin gallate, or abscisic acid; and
wherein the third SrtA inhibiting agent is different from both the first SrtA inhibiting agent and the second SrtA inhibiting agent.

3. The oral care composition of claim 1, wherein:
the sweetener comprises xylitol; and
the oral care composition contains less than 0.01 g/L sucrose.

4. An oral care composition comprising:
an orally-acceptable vehicle;
a sweetener; and
an antibiofilm-effective amount of an active compound, wherein the active compound comprises at least two of: nortrachelogenin-8′-O-β-D-glucopyranoside, lariciresinol, scopoletin, isoscopoletin, epicatechin gallate, epigallocatechin gallate, or abscisic acid.

5. The oral care composition of claim 4, wherein the orally-acceptable vehicle is aqueous, and the active compound is at a molarity concentration in a range of 20 μM to 1000 μM.

6. The oral care composition of claim 4, wherein the active compound comprises at least three of: nortrachelogenin-8′-O-β-D-glucopyranoside, lariciresinol, scopoletin, isoscopoletin, epicatechin gallate, epigallocatechin gallate, or abscisic acid.

7. The oral care composition of claim 4, wherein the active compound comprises lariciresinol, and wherein the lariciresinol is a derivative comprising secoisolariciresinol.

8. The oral care composition of claim 4, wherein the active compound comprises abscisic acid, and wherein the abscisic acid is a derivative comprising at least one of: (+) abscisic acid, (−) abscisic acid, 7-hydroxyabscisic acid, trans-abscisic acid, phaseic acid, neo-phaseic acid, or dihydrophaseic acid.

9. The oral care composition of claim 4, wherein:
the composition is formulated as a liquid mouthwash, oral spray, or oral rinse; and
the composition comprises at least one of: a buffer, a flavoring, or a surfactant.

10. The oral care composition of claim 4, wherein:
the composition is formulated as a dentifrice, toothpaste, varnish, or tooth gel; and
the composition comprises at least one of: an abrasive, a mineral source, a buffer, a flavoring, or a surfactant.

11. The oral care composition of claim 4, wherein:
the composition is formulated as a lozenge, candy, chewable tablet, gum, oral film, or gel strip; and
the composition comprises less than 1.0% sucrose by weight.

12. A cleaning composition for inhibiting biofilms, wherein the composition comprises:
a vehicle, wherein the vehicle is aqueous;
at least one of: a stabilizer, a buffer, an emulsifier, or a surfactant; and
an antibiofilm-effective amount of at least one active compound, wherein the active compound is selected from: nortrachelogenin-8′-O-β-D-glucopyranoside, lariciresinol, scopoletin, isoscopoletin, epicatechin gallate, epigallocatechin gallate, or abscisic acid.

13. The cleaning composition of claim 12, wherein the cleaning composition further comprises at least one of: polyethylene glycol, propylene glycol, glycerol, vegetable oil, citric acid, or acetic acid.

14. The cleaning composition of claim 12, wherein the composition consists entirely of non-toxic and food-safe ingredients.

15. The cleaning composition of claim 12, wherein:
the active compound is present in the cleaning composition at a molarity concentration in a range of 20.0 μM to 1000 μM.

16. The cleaning composition of claim 12, wherein the active compound degrades, inhibits, or hydrolyzes a biofilm produced by at least one bacterial strain belonging to the branch Bacillota and containing a SrtA enzyme, wherein the bacterial strain is in a genus including at least one of: Streptococcus, Staphylococcus, Enterococcus, Clostridia, Lactobacillus, or Listeria.

17. The cleaning composition of claim 12, wherein the composition is in a form selected from: a soap, a bath gel, a body scrub, a hand sanitizer, a deodorant, a facial cleanser, a wound treatment, an ear drop, a nasal spray, a topical gel, a foam, a skin lotion, a pet rinse, a cutlery rinse, a soaking solution, a fruit and vegetable rinse, a vegetable coating, a fruit wax, or a produce spray.

18. A method for making an antibiofilm composition comprising:
preparing an aqueous wood biomass extract, wherein:
the wood is maple, pecan, or hickory; and
the extract comprises an antibiofilm-effective amount of an active compound, the active compound comprising at least one of: nortrachelogenin-8′-O-B-D-glucopyranoside, lariciresinol, scopoletin, isoscopoletin, epicatechin gallate, or abscisic acid; and
combining the extract with at least one of: a buffer, a stabilizer, an emulsifier, or a surfactant.

19. The method of claim 18, wherein the active compound is from an aqueous maple extract.
